(19) United States
(12) Patent Application Publication
(10) Pub. No.: US 2026/0335036 A1
(43) Pub. Date: Oct. 29, 2026

(54) ROBOTIC SYSTEM FOR AUTOMATED SORTING AND PACKING OF SURGICAL INSTRUMENTS

(71) Applicant: RIF Robotics Corp, Atlanta, GA (US)

(72) Inventors: Collin FARILL, Atlanta, GA (US); Kevin DEMARCO, Atlanta, GA (US); Sergio GARCIA, Atlanta, GA (US)

(21) Appl. No.: 19/659,616

(22) Filed: Apr. 27, 2026

Related U.S. Application Data
(60) Provisional application No. 63/795,290, filed on Apr. 26, 2025.

(51) Int. Cl. A61B 50/33; A61B 34/32

(52) U.S. Cl. CPC A61B 50/33; A61B 34/32

(57) ABSTRACT

An instrument intake, sorting, and storage system may be provided for use with a robotic instrument handling system. The system may comprise a frame, one or more stationary racks supported by the frame, a plurality of panels configured to be passively supported on the stationary racks and to carry one or more instruments thereon, and a carriage movable relative to the frame. The carriage may comprise a plurality of fingers configured to engage engagement features of a selected panel, lift the selected panel from the stationary racks, and transport the selected panel between a plurality of operational zones, including a user-side intake zone, a robotic interaction zone, and a storage zone. By consolidating motion-producing components within the carriage, the panels and stationary racks may be substantially mechanically passive, reducing moving parts and simplifying assembly.

### CROSS-REFERENCE TO RELATED APPLICATIONS

This application claims the benefit of priority under 35 U.S.C. § 119(e) to U.S. Provisional Patent Application No. 63/795,290, titled “ROBOTIC SYSTEM FOR AUTOMATED SORTING AND PACKING OF SURGICAL INSTRUMENTS,” filed on Apr. 26, 2025, the entire contents of which are hereby incorporated by reference in their entirety for all purposes.

### TECHNICAL FIELD

The present disclosure relates generally to automated systems for handling medical devices, and more particularly to systems and apparatuses for the intake, sorting, storage, and transport of surgical instruments in connection with the assembly of surgical instrument trays. Even more particularly, the present disclosure relates to instrument intake, sorting, and storage systems comprising one or more panels configured to support surgical instruments and a transport mechanism configured to move the one or more panels between operational zones, including, for example, a user-side intake zone, a robotic interaction zone, and a storage zone.

### BACKGROUND

Surgical instrument processing may represent a critical component of healthcare operations, often requiring meticulous attention to detail, consistency, and adherence to strict protocols. Following use in surgical procedures, instruments are typically collected, cleaned, and decontaminated, and thereafter sorted and assembled into surgical trays containing specified sets of instruments needed for particular procedures. The assembled trays may then be wrapped and sterilized, such as by autoclaving, before being made available for subsequent surgical use.

Traditional manual sorting and packing of surgical instruments may be labor-intensive, prone to human error, and subject to variability in processing time and quality. Error rates in manual tray assembly have been reported to range from approximately 5% to 15% in some studies, which may lead to delays in surgical procedures, increased risk of contamination, and potential patient safety concerns. Human factors such as fatigue, distraction, and variability between shifts or between individual technicians may contribute to such errors. In addition, the manual handling of instruments may expose staff to residual contaminants and may create ergonomic concerns associated with repetitive motion over extended shifts.

Attempts have been made to automate portions of the surgical instrument processing workflow. Some existing automated systems may employ conveyor belts that transport instruments beneath a vision system, with classical robotic arms or other mechanisms attempting to identify and move the instruments. Such conveyor-based approaches may be limited in flexibility, may require relatively large footprints, and may not readily accommodate the wide variety of instrument shapes, sizes, weights, and geometries encountered in typical surgical instrument sets. Further, such systems may require instruments to be presented in a particular order or orientation, which may impose additional pre-sorting burdens on staff.

Other automated approaches may rely on fixed storage configurations in which instruments are held in predetermined positions, which may limit the ability of the system to accommodate variations in incoming instrument flow or to efficiently stage instruments between processing steps. In some such systems, instrument-supporting panels may each include integrated drive mechanisms such as wheels, tracks, motors, or similar actuators that allow the panels to move independently between locations. While such configurations may provide flexibility, they may also increase the number of moving parts in the overall system, may increase manufacturing and assembly costs, may increase maintenance requirements, and may introduce additional potential points of mechanical failure. Further, because each panel may carry its own drive components, the cost of adding storage capacity may scale unfavorably with the number of panels in the system.

Additionally, some existing automated solutions may lack robust integration between instrument identification, quality assessment, inventory tracking, and panel management functions. The absence of such integration may limit the ability of the system to, for example, identify missing or damaged instruments, locate suitable substitutes from stored inventory, and document the final tray contents in a manner suitable for regulatory and quality-assurance purposes.

Accordingly, there may be a need for automated systems and apparatuses for sorting, staging, and storing surgical instruments that may offer improved flexibility, reduced mechanical complexity, a compact footprint, and the ability to handle a wide variety of instruments presented in arbitrary order. There may also be a need for instrument intake, sorting, and storage subsystems in which panels supporting the instruments are relatively simple and substantially passive, with motion-producing components consolidated in a shared transport mechanism, so as to reduce the number of moving parts, simplify assembly, and lower overall cost while maintaining or improving operational capability.

### SUMMARY

The present disclosure provides, in at least one aspect, systems for assembling instrument trays, comprising a frame defining an intake area, a panel storage area, a robotic interaction area, and a tray area, the panel storage area comprising a plurality of storage slots arranged in a vertical stack, a plurality of panels, each panel of the plurality of panels configured to support a plurality of instruments thereon, a transport assembly supported by the frame and configured to move at least one panel of the plurality of panels among the intake area, the panel storage area, and the robotic interaction area, and a robotic arm having an end effector, the robotic arm configured to remove instruments from a panel of the plurality of panels positioned at the robotic interaction area and to arrange the removed instruments into a tray held at the tray area.

In some embodiments, each panel of the plurality of panels comprises one or more locating features configured to inhibit movement of an instrument supported on the panel relative to the panel.

In some embodiments, the one or more locating features comprise at least one of a ridge, a notch, a recess, a protrusion, or a region of compressible material disposed on or in the panel.

In some embodiments, the transport assembly is configured to receive a first panel of the plurality of panels at the intake area simultaneously with moving a second panel of the plurality of panels between two of the intake area, the panel storage area, and the robotic interaction area.

In some embodiments, the transport assembly comprises a movable element configured to engage and lift a selected panel of the plurality of panels and to translate the lifted panel relative to the frame.

In some embodiments, the plurality of storage slots are configured to passively support the plurality of panels.

In some embodiments, each panel of the plurality of panels is substantially mechanically passive.

In some embodiments, the system further comprises an identification system configured to identify one or more instruments supported on a panel of the plurality of panels positioned at the robotic interaction area.

In some embodiments, the tray area is positioned adjacent the robotic interaction area such that the robotic arm is operable to access both an instrument supported on a panel positioned at the robotic interaction area and the tray held at the tray area.

In some embodiments, the transport assembly is configured to translate at least one panel of the plurality of panels along at least a horizontal direction and a vertical direction relative to the frame.

The present disclosure provides, in at least one aspect, systems for assembling instrument trays, comprising a frame defining an intake area, a panel storage area, a robotic interaction area, and a tray area, the panel storage area comprising a plurality of storage slots arranged in a vertical stack, a plurality of panels, each panel of the plurality of panels configured to support a plurality of instruments thereon and to be received in any of the plurality of storage slots, a transport assembly configured to deliver, in a coordinated sequence, a first panel of the plurality of panels from the intake area to the robotic interaction area, to deliver the first panel from the robotic interaction area to a storage slot of the plurality of storage slots, and to deliver a second panel of the plurality of panels from another storage slot of the plurality of storage slots to the robotic interaction area, and a robotic arm having an end effector configured to transfer instruments from a panel of the plurality of panels positioned at the robotic interaction area to a tray held at the tray area.

In some embodiments, each panel of the plurality of panels comprises one or more locating features configured to inhibit movement of an instrument supported on the panel relative to the panel.

In some embodiments, the transport assembly is configured to receive a third panel of the plurality of panels at the intake area simultaneously with at least one of the deliveries by the transport assembly.

In some embodiments, the transport assembly comprises a movable element configured to engage and lift a selected panel of the plurality of panels from a storage slot of the plurality of storage slots and to translate the lifted panel between the panel storage area and at least one of the intake area and the robotic interaction area.

In some embodiments, the robotic arm comprises a multi-axis articulated arm having a plurality of degrees of freedom.

The present disclosure provides, in at least one aspect, methods of assembling an instrument tray, comprising receiving, at an intake area of a system, a first panel carrying a first plurality of instruments, moving the first panel from the intake area to a robotic interaction area of the system, transferring, by a robotic arm of the system, at least one instrument of the first plurality of instruments from the first panel to a tray held at a tray area of the system, determining that an additional instrument is to be placed into the tray, moving a second panel carrying the additional instrument from a storage slot of a panel storage area of the system to the robotic interaction area, the panel storage area comprising a plurality of storage slots arranged in a vertical stack, and transferring, by the robotic arm, the additional instrument from the second panel to the tray.

In some embodiments, the method further comprises receiving, at the intake area, a third panel carrying a third plurality of instruments simultaneously with at least one of the moving the first panel, the transferring the at least one instrument, the moving the second panel, or the transferring the additional instrument.

In some embodiments, the method further comprises identifying the at least one instrument prior to transferring the at least one instrument from the first panel.

In some embodiments, the first plurality of instruments are placed on the first panel in arbitrary positions and orientations, and wherein identifying the at least one instrument comprises estimating a position and a pose of the at least one instrument on the first panel.

In some embodiments, the method further comprises returning the first panel from the robotic interaction area to a storage slot of the plurality of storage slots after the transferring of the at least one instrument from the first panel.

Other aspects of the disclosure will become apparent by consideration of the detailed description and accompanying drawings.

### BRIEF DESCRIPTION OF THE DRAWINGS

The accompanying drawings, which are incorporated in and constitute a part of this disclosure, illustrate various embodiments of the present disclosure.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 illustrates an isometric view of an exemplary robotic system for automated sorting and packing of surgical instruments, including a robotic arm, an instrument intake, sorting, and storage system, an instrument tray and accessory area, and a user interface.

FIG. 2 illustrates an isometric view of an exemplary robotic arm and an end effector thereof positioned relative to an instrument tray area and a robotic interaction zone according to aspects of the present disclosure.

FIG. 3 illustrates another isometric view of the exemplary robotic arm and end effector illustrated in FIG. 2, shown in a different pose relative to the instrument tray area and the robotic interaction zone.

FIG. 4 illustrates an isometric view of an exemplary computer vision system positioned above the robotic arm and the robotic interaction zone according to aspects of the present disclosure.

FIG. 5 illustrates an isometric view of an exemplary instrument intake, sorting, and storage system having a two-axis movement mechanism and a plurality of panels according to a first embodiment of the present disclosure, shown with a panel positioned in a user-side intake zone.

FIG. 6 illustrates an isometric view of the exemplary instrument intake, sorting, and storage system illustrated in FIG. 5, shown with a panel positioned in a robotic interaction zone.

FIG. 7 illustrates an isometric view of an exemplary user-side intake zone and instrument tray area associated with the instrument intake, sorting, and storage system according to aspects of the present disclosure.

FIG. 8 illustrates an isometric view of an exemplary instrument intake, sorting, and storage system according to a second embodiment of the present disclosure, wherein the system comprises one or more stationary racks configured to support a plurality of substantially passive panels, and a carriage configured to engage and lift an individual panel from one of the racks and transport the lifted panel to a selected location.

FIG. 9 illustrates an enlarged isometric view of the carriage of FIG. 8, showing two pairs of fingers extending from the carriage and configured to engage a panel at corresponding engagement features to lift the panel from a rack.

FIG. 10 illustrates an isometric view of an exemplary panel of an instrument intake, sorting, and storage system according to aspects of the present disclosure, wherein the panel comprises one or more regions of compressible material configured to support a plurality of surgical instruments thereon.

FIG. 11 illustrates an isometric view of another exemplary panel of an instrument intake, sorting, and storage system according to aspects of the present disclosure, wherein the panel comprises a series of ridges, each ridge defining one or more notches, the ridges and notches together forming locating features configured to support a plurality of surgical instruments thereon.

FIG. 12 is a depiction of an exemplary flow chart illustrating a method of operating a robotic system for automated sorting and packing of surgical instruments according to aspects of the present disclosure.

### DETAILED DESCRIPTION

The present disclosure generally relates to a robotic system for the automated sorting and packing of surgical instruments, and more particularly to an instrument intake, sorting, and storage system configured to receive, stage, store, and present surgical instruments in coordination with a robotic arm for assembly of surgical instrument trays. In some embodiments, the robotic system may include a robotic arm assembly having one or more end effectors, a computer vision system configured to capture imagery of instruments for identification and quality assessment, an instrument identification and classification system configured to recognize individual instruments and evaluate their condition, an instrument intake, sorting, and storage system configured to transport instruments between operational zones, a quality control system configured to verify tray contents, a user interface and control software configured to coordinate operation of the foregoing components, and an instrument tray and accessory area configured to receive one or more surgical trays and associated organizational accessories during assembly.

While the robotic system may comprise each of the foregoing subsystems operating in cooperation with one another, the present disclosure focuses primarily on the instrument intake, sorting, and storage system and its interaction with the other subsystems of the overall robotic system. The instrument intake, sorting, and storage system may comprise one or more panels configured to support a plurality of surgical instruments, along with a transport mechanism configured to move the one or more panels, or instruments supported thereon, between two or more operational zones. The operational zones may include, for example, a user-side intake zone at which cleaned instruments may be received from a user, a robotic interaction zone at which the robotic arm may access instruments for identification, inspection, and placement into a surgical tray, and a storage zone at which one or more panels may be held pending further processing. In some embodiments, the transport mechanism may comprise a two-axis movement mechanism in which each panel includes integrated features that cooperate with corresponding structures to allow the panel to translate between zones. In other embodiments, which may be referred to herein as a simplified or reduced-complexity embodiment, the transport mechanism may comprise a carriage configured to lift and transport substantially passive panels from stationary racks, thereby consolidating moving components within the carriage and allowing the panels and racks to be mechanically simple, comparatively inexpensive to manufacture, and easier to assemble and maintain.

The instrument intake, sorting, and storage system described herein may provide enhanced flexibility in handling surgical instruments of varying shapes, sizes, weights, and geometries, may accommodate instruments presented in arbitrary order without requiring pre-sorting by a user, may provide a compact footprint suitable for a wide range of healthcare facilities, and may enable the maintenance of an on-board inventory of substitute instruments that may be drawn upon when missing or damaged instruments are identified during tray assembly. Although described herein in the context of surgical instrument processing, aspects of the disclosure may be applied to other domains involving the automated intake, sorting, staging, and storage of discrete objects, and the disclosure is not limited to surgical or medical applications unless expressly so stated.

The following detailed description is presented to enable any person skilled in the art to make and use the disclosed subject matter. For purposes of explanation, specific details are set forth to provide a thorough understanding of the present disclosure. However, it will be apparent to one skilled in the art that these specific details are not required to practice the disclosed subject matter. Descriptions of specific embodiments are provided only as representative examples. The present disclosure is not intended to be limited to the embodiments shown, but is to be accorded the widest possible scope consistent with the principles and features disclosed herein.

### Definitions

The terms “substantially” or “generally” are used to provide flexibility by recognizing that a given characteristic need not be perfectly embodied to have the desired result. Those of ordinary skill in the art will recognize that many characteristics described herein may be essentially present without strict adherence to the characteristic's definition.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “user” broadly refers to any individual who interacts with the system, including, but not limited to, operators, technicians, caregivers, and other personnel involved in the handling, inspection, sorting, packing, or processing of surgical instruments.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

A “connection” by which two components of a system, e.g., an electrical system, a data system, a computer system, a circuitry system, etc., are connected will generally be an “operable connection”, or a connection by which entities are “operably connected”. The term “operable connection” and equivalents is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, software). Logical and/or physical communication channels can be used to create an operable connection.

The term “surgical instrument,” as used herein, broadly refers to any tool, implement, or device used or intended for use in connection with a surgical, medical, dental, or veterinary procedure, including, but not limited to, scalpels, retractors, forceps, scissors, clamps, hemostats, needle holders, probes, elevators, curettes, rongeurs, and the like. The term is not limited to instruments intended for use on human subjects and may include instruments intended for use on non-human subjects.

The term “surgical tray,” as used herein, refers to a container, basket, or similar receptacle configured to hold a plurality of surgical instruments in an organized arrangement for purposes of sterilization, storage, transport, and/or presentation at the point of use. A surgical tray may include internal organizational accessories such as, but not limited to, instrument stringers, mats, bags, brackets, or silicone fingers.

The term “panel,” as used herein, refers to a substantially planar support structure configured to receive, hold, and present one or more surgical instruments. A panel may include features such as, but not limited to, slots, channels, rails, recesses, protrusions, or other locating features configured to facilitate the positioning and retention of instruments thereon and/or to facilitate pickup of instruments therefrom by a robotic end effector.

The term “locating feature,” as used herein, refers to any structure, element, or region on or associated with a panel or a tray that is configured to support, position, orient, retain, cradle, or otherwise establish a location for one or more instruments relative to the panel or tray. Locating features may include, without limitation, ridges, channels, grooves, recesses, protrusions, projections, shelves, pockets, walls, dividers, regions of compressible material, regions of resilient material, molded contours, magnetic elements, hook-and-loop elements, clips, clamps, or any combination of the foregoing. A locating feature may be rigid or deformable, may be integrally formed with the panel or tray or separately attached thereto, and may be permanently or removably associated with the panel or tray.

The term “end effector,” as used herein, refers to a device coupled to a distal portion of a robotic arm or other robotic motion system that is configured to interact with an object or the environment. An end effector may include, for example, one or more grippers, suction devices, hooks, probes, scanners, or combinations thereof.

The term “operational zone,” as used herein, refers to a defined region within or associated with the instrument intake, sorting, and storage system at which a particular operation or set of operations may be performed. Operational zones may include, for example, a user-side intake zone, a robotic interaction zone, and a storage zone, though additional or alternative zones are contemplated.

The term “transport mechanism,” as used herein, refers to any mechanical, electromechanical, or other arrangement configured to move one or more panels, or instruments supported thereon, between two or more locations. Transport mechanisms may include, for example, two-axis movement mechanisms, carriage-based lifting mechanisms, gantry systems, conveyor systems, and combinations thereof.

As used herein the terms “memory” generally refer to a physical and/or logical entity that can store data, e.g., any memory storage of a computer and is a non-transitory computer readable medium. A data store may be, for example, a database, a table, a file, a list, a queue, a heap, a memory, a register, and so on. A data store may reside in one logical and/or physical entity and/or may be distributed between multiple logical and/or physical entities. Software included in the implementation of the methods disclosed herein can be stored in the memory. The software includes, for example, firmware, one or more applications, program data, filters, rules, one or more program modules, and other executable instructions. For example, the computer can be configured to retrieve from the memory and execute, among other things, instructions related to the processes and methods described herein.

Suitable software for implementing various components of example systems and methods described herein may be developed using programming languages and tools (e.g., Java, C, C#, C++, C, SQL, APIs, SDKs, Swift, NEXTStep, SmallTalk, Unix, Objective C, assembler). Software, whether an entire system or a component of a system, may be embodied as an article of manufacture and maintained or provided as part of a computer-readable medium. Software may include signals that transmit program code to a recipient over a network or other communication medium. Thus, in one example, a computer-readable medium may be signals that represent software/firmware as it is downloaded from a server (e.g., web server).

In the foregoing description of preferred embodiments, specific terminology has been resorted to for the sake of clarity. However, the present disclosure is not intended to be limited to the specific terms so selected, and it is to be understood that each specific term includes all technical equivalents which operate in a similar manner to accomplish a similar technical purpose.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Description

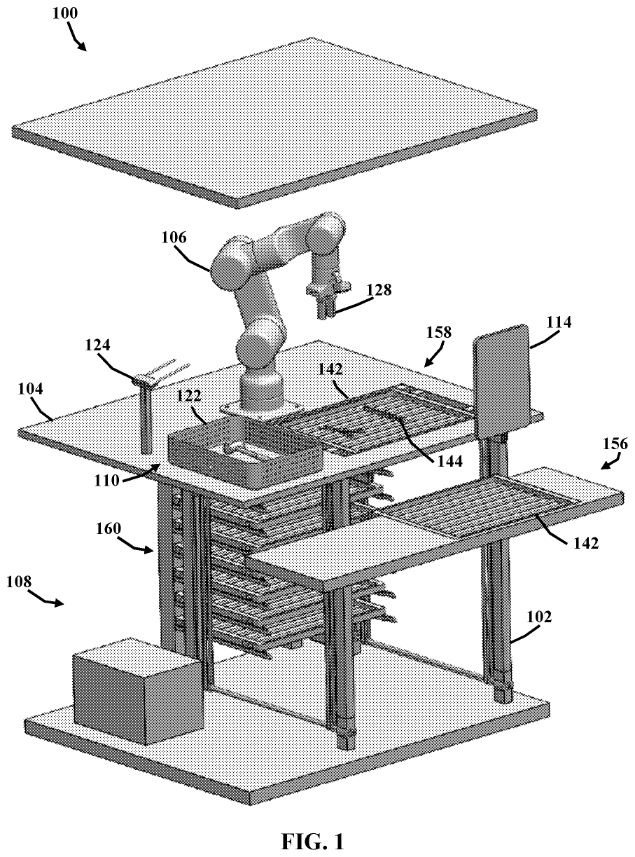

Referring now to FIG. 1, an exemplary robotic system 100 for automated sorting and packing of surgical instruments is illustrated. The robotic system 100 may comprise a frame 102, a work surface 104 supported by the frame 102, a robotic arm 106 mounted to the work surface 104, an instrument intake, sorting, and storage system 108 associated with the frame 102, an instrument tray area 110, a user interface 114, and an enclosure which may house one or more computing, power, or pneumatic components. As described in greater detail below, the instrument intake, sorting, and storage system 108 may define one or more operational zones, including a user-side intake zone 156 at which a user may load cleaned instruments 144 onto a panel 142 and a robotic interaction zone 158 at which the robotic arm 106 may access instruments 144 supported on a panel 142. In some embodiments, the user-side intake zone 156 and the robotic interaction zone 158 may be defined at portions of the work surface 104 and may serve, respectively, as the operator-accessible instrument input region and the staging region at which a panel 142 is presented for handling by the robotic arm 106. In some embodiments, the frame 102 may comprise a plurality of vertical supports and one or more horizontal cross members, which may be formed of extruded aluminum, steel, or any other suitable structural material. The work surface 104 may define a generally horizontal plane upon which a surgical tray 122 and one or more organizational accessories 124 may be placed during operation. In some embodiments, an upper panel may be disposed above the work surface 104 and may support one or more components of a computer vision system, as described in greater detail below.

The robotic arm 106 may comprise a multi-axis articulated arm having six or more degrees of freedom, such that the robotic arm 106 may be capable of positioning an end effector 128 at substantially any position and orientation within a defined workspace. In some embodiments, the robotic arm 106 may comprise a base, a plurality of arm links, a plurality of joints, and the end effector 128 coupled to a distal portion thereof. The end effector 128 may comprise, for example, a pinch-style gripper, a parallel-jaw gripper, a suction gripper, an end effector having a primary gripping mechanism and a secondary gripping mechanism, or any other suitable gripping device. In some embodiments, the end effector 128 may comprise a robotic end effector having a secondary gripping mechanism of the type described in co-pending applications, such as, for example, an end effector having a gripping panel, an appendage with an arcuate portion, and a motor configured to move the appendage transverse to a face of the gripping panel between extended and stored positions. In other embodiments, the end effector 128 may comprise any other gripper type suited to the handling of surgical instruments, including, but not limited to, three-or-more-fingered grippers, rotating-finger grippers, magnetic grippers, or combinations thereof. In some embodiments, the robotic arm 106 may further comprise a quick-change tool interface configured to allow the end effector 128 to be selectively exchanged with one or more alternative end effectors depending on the particular instrument being handled. In some embodiments, the robotic arm 106 may include force-feedback sensors, torque sensors, or collision-detection capabilities that may allow the robotic arm 106 to detect contact with an object and to limit applied force to prevent damage to delicate instruments.

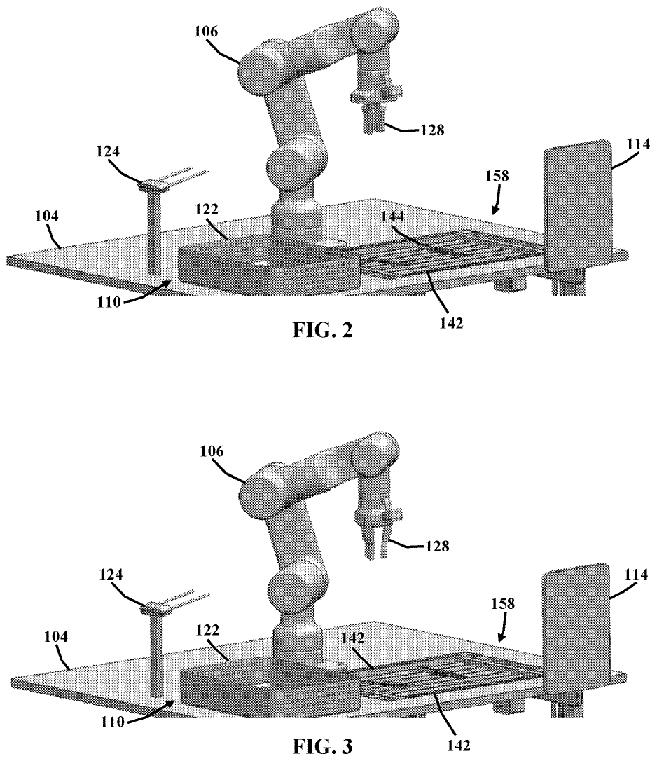

Referring now to FIGS. 2 and 3, the robotic arm 106 is illustrated in greater detail in two different poses relative to the work surface 104. A surgical tray 122 may be placed at an instrument tray area 110 defined on the work surface 104. One or more organizational accessories 124, such as an instrument stringer, may be supported at an accessory holder positioned adjacent to the instrument tray area 110. In some embodiments, the accessory holder may comprise one or more posts, brackets, clamps, or other fixtures configured to hold accessories 124 in an orientation suitable for engagement by the robotic arm 106. The robotic interaction zone 158 may be defined on the work surface 104 adjacent to the instrument tray area 110, and a panel 142 carrying one or more surgical instruments 144 may be presented at the robotic interaction zone 158 by the instrument intake, sorting, and storage system 108. A user interface 114, which may comprise a touchscreen display, may be positioned adjacent the work surface 104 to allow an operator to configure, monitor, and interact with the robotic system 100. In some embodiments, the user interface 114 may additionally or alternatively comprise physical buttons, voice-recognition inputs, gesture recognition inputs, or remote-access interfaces accessible via a network.

As shown in FIGS. 2 and 3, the robotic arm 106 may move the end effector 128 between the robotic interaction zone 158, where an instrument 144 may be picked from a panel 142, and the instrument tray area 110, where the instrument 144 may be placed into a designated position within the surgical tray 122. In some embodiments, the robotic arm 106 may approach each instrument 144 with a pose selected based on the identity, geometry, and orientation of the particular instrument 144 as determined by the computer vision system. In some embodiments, the end effector 128 may include one or more sensors, such as force/torque sensors, proximity sensors, or tactile sensors, that may be used to confirm contact with an instrument 144 or to verify that a secure grip has been achieved prior to lifting.

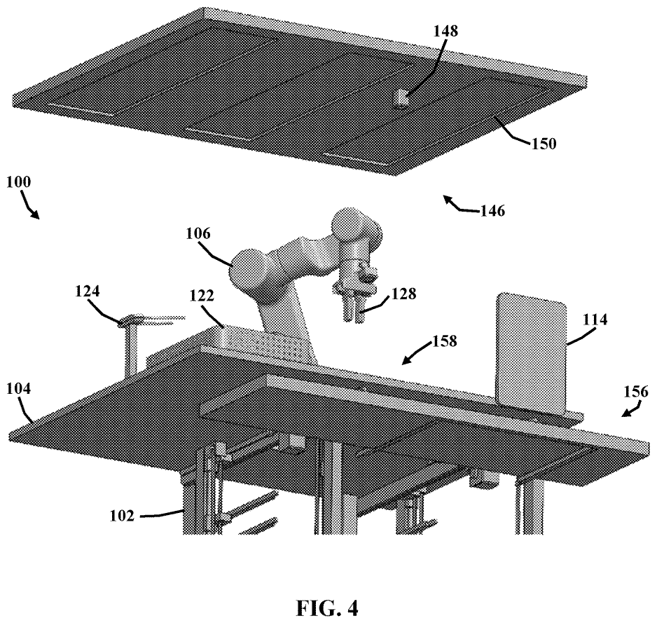

Referring now to FIG. 4, an exemplary computer vision system 146 is illustrated. The computer vision system 146 may comprise one or more cameras 148 and a lighting array 150 which may be supported by an upper panel positioned above the work surface 104, the robotic arm 106, and the robotic interaction zone 158. In some embodiments, the one or more cameras 148 may comprise high-resolution two-dimensional cameras, stereo camera pairs, depth-sensing cameras, time-of-flight sensors, structured-light sensors, or combinations thereof, which may be configured to capture imagery from a plurality of viewing angles. The lighting array 150 may comprise one or more light emitting diode (LED) modules, fluorescent sources, or other illumination sources arranged to enhance feature detection and to reduce shadows on the instruments 144 being imaged. In some embodiments, the lighting array 150 may comprise multiple independently controllable zones, which may be selectively activated to provide directional or diffused illumination depending on the reflective properties of the instrument 144 being imaged. In some embodiments, one or more additional cameras may be mounted on the robotic arm 106 or on the end effector 128 to provide close-range imagery of individual instruments 144 during handling. In some embodiments, the computer vision system 146 may further comprise real-time image processing hardware, such as one or more graphics processing units (GPUs), field-programmable gate arrays (FPGAs), or dedicated vision processors, and may include a calibration system configured to maintain measurement precision across varying operational conditions.

In some embodiments, the computer vision system 146 may cooperate with an instrument identification and classification system, which may comprise one or more deep-learning-based algorithms, convolutional neural networks, transformer-based models, or other machine-learning models configured to recognize instruments 144 regardless of orientation. The instrument identification and classification system may reference a database of instrument specifications that may include, for example, dimensions, weight, material composition, permissible handling forces, and recommended gripping locations. In some embodiments, the instrument identification and classification system may additionally detect wear, damage, residual contamination, or other defects on the instruments 144, and may track the service history of individual instruments 144 over time for lifecycle management. In some embodiments, instruments 144 may be provided with distinctive identifying features, such as, but not limited to, engraved identifiers, printed markings, radio-frequency identification (RFID) tags, or data matrix codes, that may be detected and used to uniquely identify individual instruments 144 even among visually similar items.

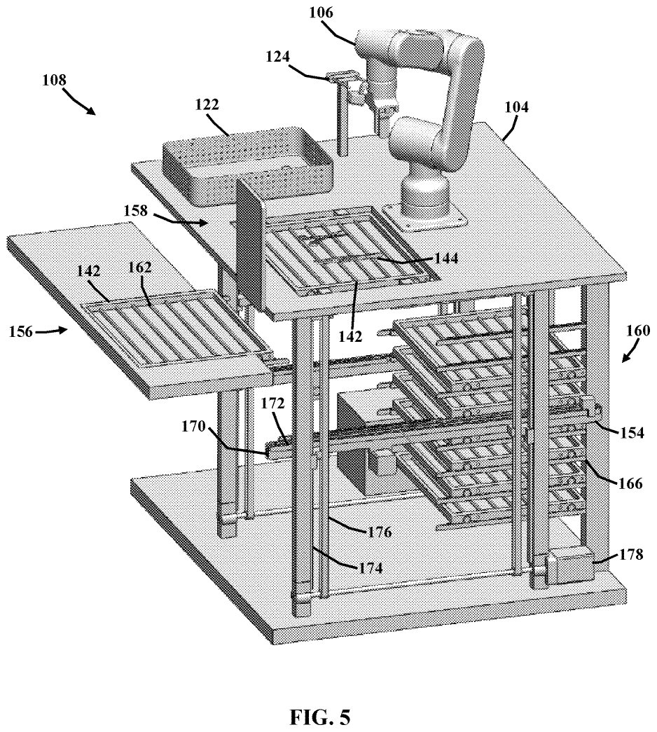

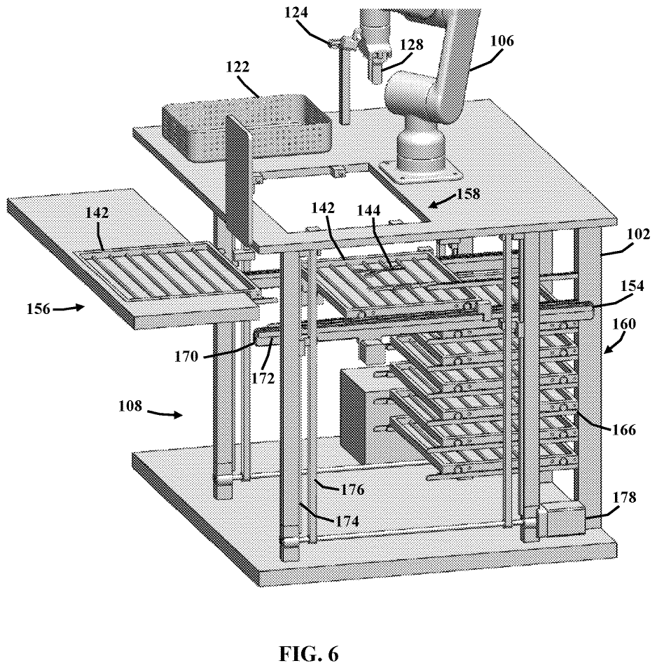

Referring now to FIG. 5, an exemplary instrument intake, sorting, and storage system 108 according to a first embodiment is illustrated. The instrument intake, sorting, and storage system 108 may comprise a plurality of panels 142, a transport mechanism 154, and a plurality of operational zones including a user-side intake zone 156, a robotic interaction zone 158, and a storage zone 160. Each panel 142 may comprise a substantially planar support structure configured to carry a plurality of surgical instruments 144. In some embodiments, instruments 144 may be placed onto a panel 142 by a user in arbitrary positions and orientations, and the computer vision system 146 may be relied upon to estimate the position and pose of each instrument 144 on the panel 142 to facilitate subsequent handling by the robotic arm 106. In some embodiments, each panel 142 may include one or more locating features configured to inhibit unintended movement of instruments 144 relative to the panel 142, to present instruments 144 in orientations suitable for identification by the computer vision system 146, and to facilitate pickup of instruments 144 by the end effector 128 of the robotic arm 106. By inhibiting rolling, sliding, or other unintended movement of instruments 144 during transport of the panel 142 between operational zones, the locating features may help maintain the position and pose of each instrument 144 between the time the panel 142 is loaded by the user and the time the instrument 144 is picked by the robotic arm 106. The locating features may be selected and arranged to address one or more challenges that may arise when a robotic end effector attempts to pick an instrument 144 from a flat surface, including, for example, difficulty in positioning a gripper finger beneath the instrument 144, unintended shifting of the instrument 144 during approach, and reduced grip security on instruments 144 having irregular, curved, or highly polished surfaces. The locating features may comprise, without limitation, a series of ridges 162, one or more regions of compressible material, one or more recesses, one or more protrusions, one or more magnetic elements, one or more hook-and-loop elements, or any combination of the foregoing, any of which may be used individually or in combination on a single panel 142. In some embodiments, each panel 142 may be configured to hold up to approximately 100 instruments 144, though panels 142 configured to hold a greater or lesser number of instruments 144 are also contemplated.

In some embodiments, and with reference to FIG. 11, the locating features may comprise a series of ridges 162 extending along at least a portion of an upper surface of the panel 142. The ridges 162 may be arranged in a substantially parallel array, in a grid pattern, in a radial pattern, or in any other pattern suitable for supporting one or more instruments 144 at elevated positions above a base surface of the panel 142. In some embodiments, each ridge 162 may be elongated in shape and may define a peak or crest configured to support an instrument 144 above the base surface of the panel 142 such that one or more channels are defined between adjacent ridges 162. The channels may provide clearance into which one or more gripper fingers, an appendage of a secondary gripping mechanism, or another portion of the end effector 128 may be inserted to pass beneath the instrument 144 during a pick operation. In some embodiments, the ridges 162 may have a cross-sectional profile that is triangular, trapezoidal, rounded, semicircular, rectangular, or any combination of the foregoing. In some embodiments, the ridges 162 may have substantially uniform height and spacing across the panel 142, while in other embodiments, the height, spacing, and orientation of the ridges 162 may vary across different regions of the panel 142 to accommodate instruments 144 of different sizes, geometries, or intended handling approaches. As shown in FIG. 11, in some embodiments one or more of the ridges 162 may further define one or more notches 164 spaced along the length of the ridge 162. Each notch 164 may comprise a localized depression, recess, or scallop in the upper edge of the ridge 162 and may be sized and shaped to receive at least a portion of an instrument 144. In some embodiments, the notches 164 of adjacent ridges 162 may be substantially aligned with one another along a direction transverse to the ridges 162, such that an elongate instrument 144 may settle laterally across the panel 142 with portions of the instrument 144 received within the aligned notches 164 of two or more adjacent ridges 162, thereby further inhibiting unintended rolling or sliding of the instrument 144 as the panel 142 is moved between operational zones. As also shown in FIG. 11, instruments 144 of differing geometries may be supported on the same panel 142 in differing relationships to the ridges 162 and notches 164. For example, an instrument 144 having a cross-sectional profile that does not nest readily within the notches 164 may rest across the upper edges of two or more ridges 162, while another instrument 144, such as a generally cylindrical instrument 144, may settle into and be cradled by one or more aligned notches 164 of adjacent ridges 162. In some embodiments, the notches 164 may be shaped, sized, and distributed to accommodate a range of instrument geometries simultaneously on a single panel 142.

In some embodiments, the ridges 162 may further assist in orienting instruments 144 upon placement onto the panel 142. For example, an instrument 144 having an elongate body, such as a scalpel, retractor, or forceps, may tend to settle longitudinally along one or more channels between adjacent ridges 162, thereby self-aligning the instrument 144 in a predictable orientation that may simplify subsequent identification by the computer vision system 146 and handling by the robotic arm 106. In some embodiments, the ridges 162 may be configured to support an instrument 144 at two or more discrete contact points along its length, which may reduce the area of contact between the instrument 144 and the panel 142 and may thereby reduce the likelihood of the instrument 144 adhering to the panel 142 due to residual moisture, surface tension, or static electricity following cleaning.

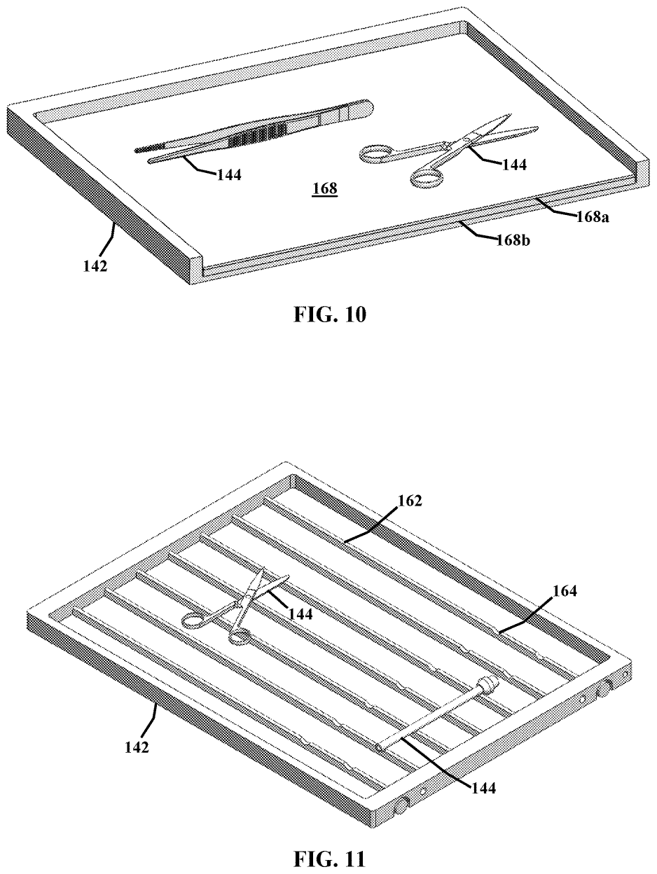

In some embodiments, and with reference to FIG. 10, the locating features may additionally or alternatively comprise one or more regions of compressible material 168 disposed on or integrated with the upper surface of the panel 142. The compressible material 168 may comprise, for example, silicone, polyurethane, thermoplastic elastomer, closed-cell or open-cell foam, neoprene, rubber, or any other suitable elastomeric or compliant material. In some embodiments, the compressible material 168 may comprise a multi-layer construction, in which two or more layers of differing material composition or differing compliance are arranged in a stacked relationship. For example, in some embodiments, the compressible material 168 may comprise an upper layer of silicone 168a disposed over a lower layer of neoprene 168b, with the silicone layer 168a providing a comparatively durable, autoclavable, and chemically resistant surface that contacts the instruments 144, and the neoprene layer 168b providing additional compliance and energy absorption beneath the silicone layer 168a. In some embodiments, the upper and lower layers may be bonded together, mechanically attached together, or simply stacked in face-to-face contact, and additional layers, including one or more reinforcement layers, one or more rigid backing layers, or one or more intermediate adhesive or interfacial layers, may be provided as appropriate. The compressible material 168 may be configured to deform locally in response to placement of an instrument 144 thereon, such that the material 168 conforms around at least a portion of the instrument 144 and thereby may nest or cradle the instrument 144 in a stable position. In some embodiments, such conforming engagement may resist unintended shifting of the instrument 144 during approach by the end effector 128 and may present the instrument 144 at a consistent and predictable pose for pickup. As shown in FIG. 10, in some embodiments, a plurality of instruments 144 of differing types, sizes, or geometries may be supported on a single region of compressible material 168 simultaneously, with the compressible material 168 deforming locally beneath each instrument 144 to accommodate that particular instrument 144.

In some embodiments, the compressible material may further be configured to deform in response to contact by the end effector 128 during a pick operation, such that one or more gripper fingers, an appendage of a secondary gripping mechanism, or another portion of the end effector 128 may press into the compressible material to a depth sufficient to pass beneath a portion of the instrument 144. By locally displacing the compressible material, the end effector 128 may form a secure grip around the instrument 144 without requiring the panel 142 to provide pre-formed clearance openings at every possible pickup location. In some embodiments, this may allow the end effector 128 to engage instruments 144 of widely varying geometries at a wide range of positions and orientations on the panel 142, while still providing firm support for the instruments 144 between picks.

In some embodiments, the compressible material may be provided as one or more pads, sheets, strips, or molded elements attached to or embedded within the panel 142, and may cover all or only a portion of the upper surface of the panel 142. In some embodiments, the compressible material may define one or more recessed regions shaped to correspond to particular instrument types, with the recessed regions serving both to locate the instruments 144 and to present unobstructed edges along which the end effector 128 may approach. In some embodiments, the compressible material may be overmolded onto or bonded to a rigid substrate forming the structural body of the panel 142, while in other embodiments, the compressible material may be removable and replaceable, allowing worn or contaminated material to be exchanged without replacing the entire panel 142. In some embodiments, the compressible material may be autoclavable, chemically resistant to cleaning agents, and otherwise compatible with the sterilization and processing environments in which the robotic system 100 may be used.

In some embodiments, the locating features may comprise any combination of the foregoing configurations. For example, a panel 142 may comprise a plurality of rigid ridges 162 interspersed with regions of compressible material, such that instruments 144 may be supported on the ridges 162 while portions of the compressible material provide additional conforming support and locally deformable surfaces for the end effector 128 to engage. In other embodiments, the ridges 162 themselves may be formed of, or may be coated or capped with, a compressible material, such that the ridges 162 may provide elevated support for the instruments 144 while also being capable of local deformation during handling. In some embodiments, the panel 142 may further comprise magnetic elements, hook-and-loop fasteners, or other retention features configured to cooperate with corresponding features on selected instruments 144 or on the ridges 162 or the compressible material to provide additional securement during transport of the panel 142 between operational zones.

In some embodiments, the surgical tray 122 itself may include one or more tray surface features that correspond to, or share characteristics with, the locating features of the panels 142. For example, the surgical tray 122 may include one or more ridges, one or more regions of compressible material, one or more molded recesses, or combinations thereof, that may be configured to receive instruments 144 in predetermined positions and to permit the end effector 128 to release instruments 144 into the tray 122 without interference. In some embodiments, such tray surface features may also be configured to deform locally around placed instruments 144 to cradle and secure the instruments 144 during subsequent wrapping, handling, and sterilization of the tray 122. In some embodiments, a control system of the robotic system 100 may coordinate selection of a panel 142 having a particular locating feature configuration with selection of a tray 122 having a corresponding tray surface feature configuration, such that the instruments 144 are consistently supported and presented throughout the pickup, transport, and placement operations.

The user-side intake zone 156 may be positioned on a user-accessible side of the instrument intake, sorting, and storage system 108 and may be configured to receive one or more panels 142 upon which a user has placed cleaned instruments 144. In some embodiments, an intake extension may extend outwardly from the frame 102 to provide a convenient position at which a user may load instruments 144 onto a panel 142. The robotic interaction zone 158 may be positioned within reach of the robotic arm 106, and may be configured to present a selected panel 142 at a location and orientation suitable for the robotic arm 106 to access individual instruments 144 thereon. The storage zone 160 may be configured to hold one or more panels 142 that are not currently being filled, unloaded, or processed. In some embodiments, the storage zone 160 may be organized as a vertical array of storage slots 166 arranged on one or both sides of the transport mechanism 154, thereby making efficient use of three-dimensional space within the system 100.

In the first embodiment shown in FIG. 5, the transport mechanism 154 may comprise a two-axis movement mechanism configured to move a panel 142 horizontally between operational zones and to move the transport mechanism 154 itself vertically between vertical positions corresponding to the robotic interaction zone 158 and each of the storage slots 166. In some embodiments, the transport mechanism 154 may comprise a pair of generally parallel arms 170 that extend in a horizontal direction and that are spaced apart from one another by a distance corresponding generally to a width of a panel 142. Each arm 170 of the pair may carry, define, or otherwise support one or more horizontal tracks 172 oriented along the length of the arm 170. The horizontal tracks 172 may comprise rails, grooves, channels, slotted guides, roller paths, drive surfaces, or any combination thereof, and may be configured to engage corresponding panel-side drive features, such as wheels, rollers, tracks, lugs, pins, or the like, provided on opposite lateral edges of each panel 142. With a panel 142 received between the pair of arms 170 and supported on the horizontal tracks 172, the panel 142 may translate horizontally along the arms 170 between, for example, the user-side intake zone 156 at one end of the pair of arms 170 and the robotic interaction zone 158 at the other end, or between either of those zones and a position aligned with a selected storage slot 166. In some embodiments, horizontal motion of the panel 142 along the horizontal tracks 172 may be powered by panel-integrated drive features that engage the horizontal tracks 172, by drive elements integrated into the arms 170 themselves, by an external pusher or actuator coupled to the panel 142, or by any combination of the foregoing.

In some embodiments, the pair of arms 170, together with the horizontal tracks 172 carried thereon, may collectively form a horizontally translating platform that is itself movable in a vertical direction relative to the frame 102. To this end, the transport mechanism 154 may further comprise a pair of generally parallel vertical tracks 174 supported by the frame 102, the vertical tracks 174 being spaced apart from one another such that the pair of arms 170 may be supported at corresponding ends thereof for guided vertical motion along the vertical tracks 174. The vertical tracks 174 may comprise linear rails, linear bearings, guide rods, channeled members, or any combination thereof. In some embodiments, the transport mechanism 154 may further comprise one or more belts 176 routed along the vertical tracks 174 and operably coupled to one or both ends of the pair of arms 170, such that movement of the belts 176 produces corresponding vertical movement of the pair of arms 170 along the vertical tracks 174. In some embodiments, the one or more belts 176 may comprise toothed timing belts, while in other embodiments the belts 176 may be replaced or supplemented by chains, cables, lead screws, ball screws, rack-and-pinion drives, or other suitable linear-motion components. A motor 178, which may comprise an electric motor, a stepper motor, a servomotor, or any other suitable rotary actuator, may be supported by the frame 102 and may be operably coupled to the one or more belts 176, such that operation of the motor 178 drives the belts 176 and thereby drives the pair of arms 170 vertically along the vertical tracks 174 between selected vertical positions. In some embodiments, a single motor 178 may drive both belts 176 in synchrony, while in other embodiments separate motors 178 may be provided for each belt 176, with control electronics coordinating their operation to maintain the pair of arms 170 in a substantially level orientation as they travel.

By coordinating vertical movement of the pair of arms 170 along the vertical tracks 174 with horizontal movement of a panel 142 along the horizontal tracks 172, the transport mechanism 154 may move any selected panel 142 between the user-side intake zone 156, the robotic interaction zone 158, and any one of the plurality of storage slots 166 within the storage zone 160. For example, to retrieve a panel 142 from a particular storage slot 166, the motor 178 may drive the belts 176 to position the pair of arms 170 at a vertical height aligned with that storage slot 166, after which the panel 142 may be drawn horizontally along the horizontal tracks 172 from the storage slot 166 onto the pair of arms 170. The pair of arms 170 may then be raised or lowered along the vertical tracks 174 to a vertical height aligned with the robotic interaction zone 158, and the panel 142 may be advanced horizontally along the horizontal tracks 172 to the robotic interaction zone 158. A reverse sequence may be used to return a panel 142 from the robotic interaction zone 158 to a storage slot 166. In some embodiments, the storage zone 160 may comprise a plurality of storage slots 166 arranged as a vertical array on one side of the pair of arms 170, on two opposing sides of the pair of arms 170, or otherwise distributed about the travel path of the transport mechanism 154. In some embodiments, each storage slot 166 may comprise a pair of opposed support rails, ledges, brackets, or other passive support features sized and positioned to receive a panel 142 transferred horizontally from the pair of arms 170, such that a panel 142 may be transferred between the pair of arms 170 and a selected storage slot 166 by way of horizontal translation along the horizontal tracks 172. In some embodiments, the user-side intake zone 156 and the robotic interaction zone 158 may similarly be defined at fixed vertical positions relative to the frame 102, with corresponding support features at those positions configured to cooperate with the horizontal tracks 172 of the pair of arms 170 to receive a panel 142 transferred horizontally to or from the pair of arms 170. In some embodiments, the horizontal travel of the panels 142 may be longer along one axis than along another, and panels 142 may be configured to be engaged, lifted, or released from a consistent side, which may simplify control logic and reduce the mechanical complexity of interfaces with the panels 142.

Referring now to FIG. 6, the instrument intake, sorting, and storage system 108 of the first embodiment is illustrated with a panel 142 positioned in the robotic interaction zone 158. In this configuration, the panel 142 may be presented at a height and orientation that permits the robotic arm 106 to efficiently access instruments 144 thereon. In some embodiments, once the robotic arm 106 has removed all needed instruments 144 from the panel 142, the transport mechanism 154 may return the panel 142 to the storage zone 160 and may retrieve an additional panel 142 from the storage zone 160 or the user-side intake zone 156 for further processing. In some embodiments, the instrument intake, sorting, and storage system 108 may maintain a digital inventory of the instruments 144 present on each panel 142 and the location of each panel 142 within the system, and may select the next panel 142 to present to the robotic arm 106 based on the content of a current surgical tray configuration being assembled.

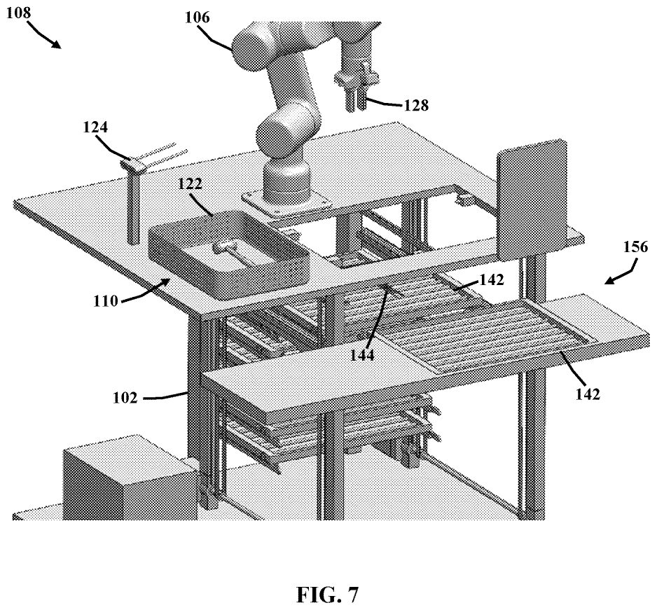

Referring now to FIG. 7, an exemplary user-side intake zone and instrument tray area are illustrated. In some embodiments, the user-side intake zone 156 may be positioned on a user-accessible side of the frame 102 and may include or be configured to receive one or more panels 142 presented in a manner that permits a user to place cleaned instruments 144 thereon. The user-side intake zone 156 may thus serve as the operator-accessible instrument input region of the system 100, and references herein to an operator input area or to the loading of instruments 144 by a user may be understood to refer to the user-side intake zone 156. In some embodiments, an instrument tray 122 may be placed at the instrument tray area 110 on an opposite side or on the same side of the frame 102, and one or more accessory holders may be provided to hold organizational accessories 124 used in the assembly of the tray 122. In some embodiments, the user-side intake zone 156 and the instrument tray area 110 may be positioned such that a user may load instruments 144 into the system 100 while the robotic arm 106 simultaneously assembles a tray 122 at the instrument tray area 110, thereby permitting parallel human and robotic operation.

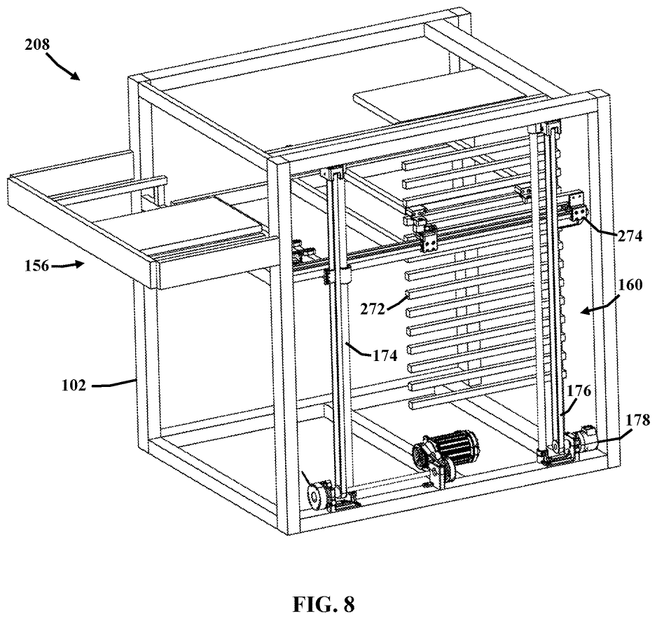

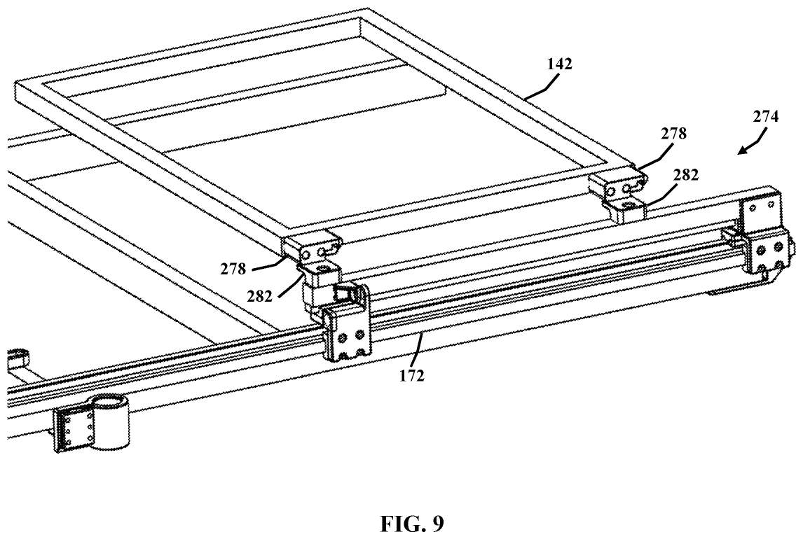

Referring now to FIG. 8, an exemplary instrument intake, sorting, and storage system 208 according to a second embodiment of the present disclosure is illustrated. The instrument intake, sorting, and storage system 208 according to the second embodiment may comprise a frame 102, one or more stationary racks 272 supported by the frame 102, a plurality of panels 142 supported on the one or more stationary racks 272, a carriage 274 movable relative to the stationary racks 272, and a plurality of operational zones including a user-side intake zone 156, a robotic interaction zone 158, and a storage zone 160. Components of the second embodiment that function similarly to components of the first embodiment may share the same reference numerals, and certain components unique to the second embodiment are identified with additional reference numerals introduced below. For purposes of clarity, FIGS. 8 and 9 depict only selected portions of the second embodiment, and certain components of the system 208 are omitted from these figures. For example, the upper portion of the system 208, which may include the work surface 104, the robotic arm 106, the instrument tray area 110, the surgical tray 122, and one or more instruments 144, is not shown in FIGS. 8 and 9. In addition, FIGS. 8 and 9 illustrate only a portion of the rack arrangement on one side of the carriage 274, and it should be understood that, in some embodiments, corresponding stationary racks 272 and corresponding engagement features 278 of the panels 142 may be provided on the opposite side of the carriage 274, with corresponding fingers 282 of the carriage 274 extending toward those features. The omitted features are described elsewhere herein and may be employed in conjunction with the second embodiment as appropriate.

In the second embodiment, the stationary racks 272 may be substantially mechanically passive, in that the racks 272 themselves may not include drive motors, wheels, rollers, or other actuators. Rather, each stationary rack 272 may comprise one or more support features, such as shelves, ledges, brackets, pins, or rails, configured to passively support one or more panels 142 at defined vertical and horizontal positions. Similarly, each panel 142 of the second embodiment may be substantially mechanically passive, in that the panel 142 itself may not include integrated drive motors, wheels, or tracks. Each panel 142 may comprise a substantially planar support structure configured to carry a plurality of surgical instruments 144, and may include one or more locating features for receiving and retaining instruments 144, which may include any of the locating feature configurations described above with respect to the panels 142 of the first embodiment. For example, the locating features may comprise a series of ridges 162, one or more regions of compressible material, one or more recesses, one or more protrusions, one or more magnetic elements, one or more hook-and-loop elements, or any combination of the foregoing, configured to support instruments 144 at elevated positions, to self-orient elongate instruments 144 into predictable positions, to deform around or locally beneath instruments 144 during placement or pickup, or any combination of the foregoing. Because the panels 142 of the second embodiment may be substantially mechanically passive and may be formed at relatively low cost, in some embodiments a plurality of panels 142 having different locating feature configurations may be provided with the system 208, and the carriage 274 may selectively retrieve a panel 142 having a locating feature configuration suited to the particular instruments 144 being processed at a given time. In some embodiments, an identifier, such as a printed marking, an engraved feature, or an RFID tag, may be associated with each panel 142 to allow the control system to track the locating feature configuration of the panel 142 and to select panels 142 accordingly.

Each panel 142 may further include one or more engagement features 278 configured to be engaged by the carriage 274 to allow the panel 142 to be lifted from the stationary rack 272. The engagement features 278 may comprise, for example, slots, recesses, flanges, holes, notches, undercuts, or any other features suitable for engagement from below or from a side of the panel 142.

Because the panels 142 and the stationary racks 272 of the second embodiment may be mechanically passive, and because motion-producing components may be consolidated within the carriage 274, the overall number of moving parts within the system 208 may be reduced relative to the first embodiment. This may simplify manufacturing, reduce the cost of adding additional storage capacity (since adding storage capacity may involve adding only additional passive panels 142 and, if needed, additional passive racks 272), and reduce maintenance requirements over the operational life of the system 208.

Referring now to FIG. 9, an enlarged view of the carriage 274 is illustrated. The carriage 274 may comprise a carriage body supported for movement along one or more axes relative to the frame 102, and two pairs of fingers 282 extending from the carriage body. The two pairs of fingers 282 may be spaced apart from one another along a first direction and may extend generally parallel to a second direction, such that the two pairs of fingers 282 are positioned to engage corresponding engagement features 278 on opposite sides or corners of a panel 142 (shown as just the outer rectangular frame without the base portion). In some embodiments, each finger 282 may comprise an elongate rigid member, which may be formed of metal, a rigid polymer, or another suitable material, and may be sized and shaped to be inserted beneath, into, or alongside an engagement feature 278 of a panel 142. In some embodiments, the fingers 282 may be fixed relative to the carriage body, such that engagement of a panel 142 may be achieved through vertical motion of the carriage 274 relative to the stationary rack 272. In other embodiments, the fingers 282 may be selectively extendable and retractable relative to the carriage body, such that the fingers 282 may be retracted during translation of the carriage 274 and extended to engage an engagement feature 278 at a desired position.

In some embodiments, the carriage 274 may be movable along a horizontal axis and a vertical axis relative to the frame 102, which may be achieved by, for example, one or more linear guides, lead screws, belt drives, chain drives, rack-and-pinion drives, or combinations thereof, powered by one or more motors mounted on or associated with the carriage 274 or the frame 102. In other embodiments, the carriage 274 may be movable along additional axes, including, for example, a second horizontal axis orthogonal to the first horizontal axis, or a rotational axis. To transport a panel 142 between locations, the carriage 274 may be aligned with a selected panel 142, raised or lowered such that the fingers 282 engage the engagement features 278 of the panel 142, lifted further to separate the panel 142 from the support features of the stationary rack 272, translated to a destination location corresponding to another operational zone or another location within the storage zone 160, and then lowered such that the panel 142 is deposited onto corresponding support features at the destination. The carriage 274 may then disengage from the panel 142 by further vertical motion or by retracting the fingers 282.

In some embodiments, the carriage 274 may include one or more sensors, such as position sensors, load sensors, optical sensors, or proximity sensors, configured to verify that a panel 142 has been properly engaged prior to lifting, to confirm the presence or absence of a panel 142 at a given rack position, or to monitor the weight of a panel 142 in order to detect unexpected conditions, such as an instrument 144 having been left in an incorrect location. In some embodiments, the carriage 274 may also include a camera or other imaging device to assist in aligning the fingers 282 with the engagement features 278 of a panel 142. In some embodiments, the carriage 274 may be configured to simultaneously carry only a single panel 142, while in other embodiments the carriage 274 may be configured to simultaneously carry two or more panels 142 stacked or otherwise arranged relative to the carriage body.

In some embodiments, the stationary racks 272 of the second embodiment may be arranged on one side of the carriage 274, on two opposing sides of the carriage 274, or on three or more sides surrounding a travel path of the carriage 274. In some embodiments, the user-side intake zone 156 may comprise a position at which the carriage 274 may place a panel 142 such that the panel 142 is accessible to a user from outside the frame 102. In some embodiments, the robotic interaction zone 158 may comprise a position at which the carriage 274 may place a panel 142 within reach of the robotic arm 106. In some embodiments, the user-side intake zone 156 and the robotic interaction zone 158 may comprise dedicated surfaces or fixtures distinct from the stationary racks 272, while in other embodiments, one or more of the operational zones may comprise a designated portion of the stationary racks 272.

In operation, the robotic system 100 may perform a method for automated sorting and packing of surgical instruments 144. Cleaned instruments 144 may initially be placed by a user onto one or more panels 142 at the user-side intake zone 156. The transport mechanism 154, or in the second embodiment the carriage 274, may transport a loaded panel 142 to the robotic interaction zone 158. The computer vision system 146 may acquire imagery of the panel 142 and the instruments 144 thereon, and the instrument identification and classification system may determine the identity, pose, condition, and any distinguishing features of each instrument 144. In some embodiments, instruments 144 that fail one or more inspection criteria, such as indications of residual contamination, damage, or wear beyond an acceptable threshold, may be flagged for manual review and may be handled differently than instruments that pass the inspection criteria, such as by being placed into a segregation area rather than onto the surgical tray 122.

A control system of the robotic system 100 may determine an optimal picking sequence based on a target surgical tray configuration and the identified instruments 144 available on one or more panels 142. The robotic arm 106 may then approach each selected instrument 144 with a pose, grip configuration, and applied force suited to the characteristics of that particular instrument 144, may transfer the instrument 144 to its designated position within the surgical tray 122, and may release the instrument 144. In some embodiments, one or more placement sensors, which may comprise force/torque sensors, optical sensors, or cameras, may be used to confirm proper positioning of the instrument 144 within the tray 122. In some embodiments, the computer vision system 146 may be used to acquire a verification image of each placed instrument 144, which may be stored along with other process data as part of a detailed packing record for regulatory, quality-assurance, or inventory-management purposes.

Once the robotic arm 106 has completed placement of the scheduled instruments 144, the control system may perform a final verification scan of the surgical tray 122 using the computer vision system 146 to confirm completeness. If one or more instruments 144 are determined to be missing, damaged, or otherwise unsuitable, the control system may query the on-board inventory of panels 142 within the storage zone 160 to identify one or more panels 142 carrying suitable substitute instruments 144, and the transport mechanism 154 or carriage 274 may retrieve the identified panel 142 to the robotic interaction zone 158 for processing. The robotic arm 106 may then pick and place the substitute instrument 144, and the control system may update the packing record accordingly. Once the surgical tray 122 has been verified as complete, the completed tray 122 may be released for wrapping, sterilization (e.g., via autoclaving, or other forms of sterilization), and subsequent use in an operating room. In some embodiments, process data generated during assembly of the tray 122 may be transmitted to one or more central inventory management systems, hospital information systems, cloud-based servers, or other external platforms.

The robotic system 100 may be deployed in a variety of settings, including, but not limited to, hospital central sterile processing departments, offsite sterile processing centers, ambulatory surgical centers, dental facilities, veterinary surgical facilities, military field hospitals, and medical device manufacturing facilities. In some embodiments, the robotic system 100 may be provided as a fixed installation, while in other embodiments, the robotic system 100 may be provided as a mobile cart-based system that may be relocated within a facility, or as a compact version suited for smaller facilities. In some embodiments, the robotic system 100 may operate in a standalone configuration, while in other embodiments, the robotic system 100 may be integrated with existing hospital information systems, enterprise resource planning systems, inventory management systems, or cloud-based management platforms supporting operations across multiple facilities. In some embodiments, specialized versions of the robotic system 100 may be provided for particular surgical specialties, such as, for example, an orthopedic-focused configuration capable of handling implant components, a microsurgery-focused configuration with enhanced precision for delicate instruments, or an endoscopic-focused configuration with specialized handling for flexible instruments.

In some embodiments, one or more of the robotic arm 106, the computer vision system 146, the instrument identification and classification system, the instrument intake, sorting, and storage system 108, 208, the quality control system, the user interface 114, and any other subsystems of the robotic system 100 may be operably connected to, and controlled by, a computing device, which may be of any suitable type. The computing device may comprise, for example, a desktop computer, a server, an embedded computer, an industrial controller, a laptop, a tablet, or any combination thereof, and may include one or more processors, memory, non-volatile storage, and communication interfaces configured to exchange data with the various subsystems and with external systems as described above. In some embodiments, the computing device may implement one or more machine-learning models that may be updated over time based on operational data collected from the robotic system 100, thereby allowing the performance of the system 100 to improve with use. In some embodiments, the computing device may support remote monitoring and remote operation of the robotic system 100 via a network connection, which may enable supervisory personnel located at a remote site to observe system status, respond to alerts, or initiate interventions as needed.

Although the instrument intake, sorting, and storage system 108, 208 has been described herein primarily in the context of surgical instrument processing, aspects of the disclosed system may be applied to other domains involving the automated intake, sorting, staging, and storage of discrete objects, including, but not limited to, industrial parts kitting, pharmaceutical compounding, laboratory sample handling, and electronic component assembly. The particular size, shape, arrangement, and number of panels 142, operational zones 156, 158, 160, stationary racks 272, and other features described herein are illustrative only, and a person of ordinary skill in the art will appreciate that numerous modifications, substitutions, and rearrangements are possible without departing from the scope of the present disclosure.

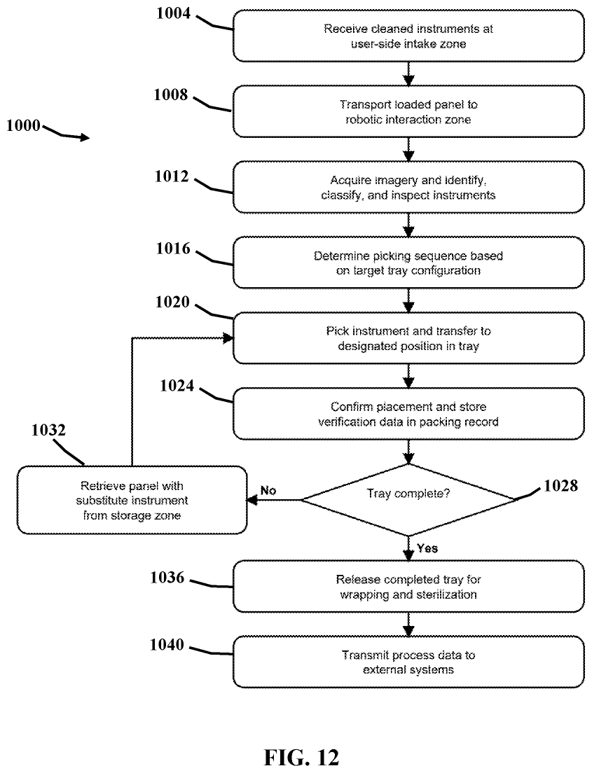

Referring now to FIG. 12, an exemplary method 1000 of operating the robotic system 100 is illustrated in flowchart form. At step 1004, one or more cleaned surgical instruments 144 may be received at the user-side intake zone 156. In some embodiments, a user may place the cleaned instruments 144 onto a panel 142 at the user-side intake zone 156 following a cleaning operation, such as a washer-disinfector cycle. At step 1008, the transport mechanism 154, or in the second embodiment the carriage 274, may transport the loaded panel 142 from the user-side intake zone 156 to the robotic interaction zone 158. At step 1012, the computer vision system 146 may acquire imagery of the panel 142 and the one or more instruments 144 thereon, and the instrument identification and classification system may determine the identity, pose, condition, and any distinguishing features of each instrument 144. In some embodiments, step 1012 may further comprise determining whether one or more instruments 144 satisfy one or more inspection criteria, such as cleanliness, integrity, or the absence of wear or damage beyond a predetermined threshold.

At step 1016, a control system may determine a picking sequence based on a target surgical tray configuration and the identified instruments 144 available on one or more panels 142. In some embodiments, the picking sequence may be optimized to minimize total travel distance of the robotic arm 106, to minimize total cycle time, to minimize the number of panel changes, or based on any other suitable criterion or combination of criteria. At step 1020, the robotic arm 106 may approach a selected instrument 144 with a pose, grip configuration, and applied force suited to the characteristics of that particular instrument 144, may pick the instrument 144 from the panel 142, and may transfer the instrument 144 to its designated position within the surgical tray 122. At step 1024, the control system may confirm proper placement of the instrument 144 within the surgical tray 122 using one or more placement sensors, the computer vision system 146, or a combination thereof, and may store a verification image or other verification data as part of a packing record. Steps 1020 and 1024 may be repeated for each additional instrument 144 designated by the picking sequence.

At step 1028, the control system may determine whether the surgical tray 122 has been completed in accordance with the target surgical tray configuration. If one or more instruments 144 are determined to be missing, damaged, or otherwise unsuitable, the method 1000 may proceed to step 1032, at which the control system may query an on-board inventory of panels 142 within the storage zone 160 to identify one or more panels 142 carrying suitable substitute instruments 144. The transport mechanism 154 or carriage 274 may retrieve the identified panel 142 to the robotic interaction zone 158, and steps 1020 and 1024 may be repeated for the substitute instrument 144. If the surgical tray 122 has been completed, the method 1000 may proceed to step 1036, at which the completed surgical tray 122 may be released for wrapping, sterilization (e.g., via autoclaving or other form of sterilization), and subsequent use in an operating room. At step 1040, process data generated during assembly of the tray 122 may be transmitted to one or more external systems, such as a central inventory management system, a hospital information system, or a cloud-based management platform. In some embodiments, the order of the steps of method 1000 may be varied, one or more of the steps may be performed in parallel, and one or more steps may be added, omitted, or combined without departing from the scope of the present disclosure. For example, in some embodiments, additional panels 142 may be received at the user-side intake zone 156 simultaneously with the performance of one or more of steps 1012 through 1028, such that user input of cleaned instruments 144 may proceed in parallel with ongoing tray assembly operations.

Various features and advantages are set forth in the following claims.

What is claimed is:

1. A system for assembling instrument trays, comprising:
a frame defining an intake area, a panel storage area, a robotic interaction area, and a tray area, the panel storage area comprising a plurality of storage slots arranged in a vertical stack;
a plurality of panels, each panel of the plurality of panels configured to support a plurality of instruments thereon;
a transport assembly supported by the frame and configured to move at least one panel of the plurality of panels among the intake area, the panel storage area, and the robotic interaction area; and
a robotic arm having an end effector, the robotic arm configured to remove instruments from a panel of the plurality of panels positioned at the robotic interaction area and to arrange the removed instruments into a tray held at the tray area.

2. The system of claim 1, wherein each panel of the plurality of panels comprises one or more locating features configured to inhibit movement of an instrument supported on the panel relative to the panel.

3. The system of claim 2, wherein the one or more locating features comprise at least one of a ridge, a notch, a recess, a protrusion, or a region of compressible material disposed on or in the panel.

4. The system of claim 1, wherein the transport assembly is configured to receive a first panel of the plurality of panels at the intake area simultaneously with moving a second panel of the plurality of panels between two of the intake area, the panel storage area, and the robotic interaction area.

5. The system of claim 1, wherein the transport assembly comprises a movable element configured to engage and lift a selected panel of the plurality of panels and to translate the lifted panel relative to the frame.

6. The system of claim 1, wherein the plurality of storage slots are configured to passively support the plurality of panels.

7. The system of claim 1, wherein each panel of the plurality of panels is substantially mechanically passive.

8. The system of claim 1, further comprising an identification system configured to identify one or more instruments supported on a panel of the plurality of panels positioned at the robotic interaction area.

9. The system of claim 1, wherein the tray area is positioned adjacent the robotic interaction area such that the robotic arm is operable to access both an instrument supported on a panel positioned at the robotic interaction area and the tray held at the tray area.

10. The system of claim 1, wherein the transport assembly is configured to translate at least one panel of the plurality of panels along at least a horizontal direction and a vertical direction relative to the frame.

11. A system for assembling instrument trays, comprising:
a frame defining an intake area, a panel storage area, a robotic interaction area, and a tray area, the panel storage area comprising a plurality of storage slots arranged in a vertical stack;
a plurality of panels, each panel of the plurality of panels configured to support a plurality of instruments thereon and to be received in any of the plurality of storage slots;
a transport assembly configured to deliver, in a coordinated sequence, a first panel of the plurality of panels from the intake area to the robotic interaction area, to deliver the first panel from the robotic interaction area to a storage slot of the plurality of storage slots, and to deliver a second panel of the plurality of panels from another storage slot of the plurality of storage slots to the robotic interaction area; and
a robotic arm having an end effector configured to transfer instruments from a panel of the plurality of panels positioned at the robotic interaction area to a tray held at the tray area.

12. The system of claim 11, wherein each panel of the plurality of panels comprises one or more locating features configured to inhibit movement of an instrument supported on the panel relative to the panel.

13. The system of claim 11, wherein the transport assembly is configured to receive a third panel of the plurality of panels at the intake area simultaneously with at least one of the deliveries by the transport assembly.

14. The system of claim 11, wherein the transport assembly comprises a movable element configured to engage and lift a selected panel of the plurality of panels from a storage slot of the plurality of storage slots and to translate the lifted panel between the panel storage area and at least one of the intake area and the robotic interaction area.

15. The system of claim 11, wherein the robotic arm comprises a multi-axis articulated arm having a plurality of degrees of freedom.

16. A method of assembling an instrument tray, comprising:
receiving, at an intake area of a system, a first panel carrying a first plurality of instruments;
moving the first panel from the intake area to a robotic interaction area of the system;
transferring, by a robotic arm of the system, at least one instrument of the first plurality of instruments from the first panel to a tray held at a tray area of the system;
determining that an additional instrument is to be placed into the tray;
moving a second panel carrying the additional instrument from a storage slot of a panel storage area of the system to the robotic interaction area, the panel storage area comprising a plurality of storage slots arranged in a vertical stack; and
transferring, by the robotic arm, the additional instrument from the second panel to the tray.

17. The method of claim 16, further comprising receiving, at the intake area, a third panel carrying a third plurality of instruments simultaneously with at least one of the moving the first panel, the transferring the at least one instrument, the moving the second panel, or the transferring the additional instrument.

18. The method of claim 16, further comprising identifying the at least one instrument prior to transferring the at least one instrument from the first panel.

19. The method of claim 18, wherein the first plurality of instruments are placed on the first panel in arbitrary positions and orientations, and wherein identifying the at least one instrument comprises estimating a position and a pose of the at least one instrument on the first panel.

20. The method of claim 16, further comprising returning the first panel from the robotic interaction area to a storage slot of the plurality of storage slots after the transferring of the at least one instrument from the first panel.
